(19) United States
(12) Patent Application Publication
(10) Pub. No.: US 2010/0274329 A1
(43) Pub. Date: Oct. 28, 2010

(54) SYSTEM AND METHOD FOR SKIN CARE USING LIGHT AND MICROCURRENTS

(76) Inventors: Chris Bradley, Orinda, CA (US); Kate Somerville, Los Angeles, CA (US); Jeffrey Tilley, Le Honda, CA (US)

Correspondence Address: Cislo & Thomas LLP, 1333 2nd Street, Suite #500, Santa Monica, CA, 90401-4110, US

(21) Appl. No.: 12/429,935

(22) Filed: Apr. 24, 2009

(51) Int. Cl. A61N 5/06; A61N 1/04

(52) U.S. Cl. 607/90; 607/115

(57) ABSTRACT

Apparatus, device, system, kit, and method to treat and prevent skin conditions. The device incorporates two treatment technologies: photorejuvenation and microcurrents. The photorejuvenation technology incorporates the use of light emitting diodes to treat skin blemishes. The light emitting diodes typically emit light having a wavelength of about 630 nanometers. The device also incorporates the use of low level microcurrent pulses to tighten muscles and skin. The electrodes are placed in a concentric circle pattern. The pattern allows for current to flow evenly from a cathode to anode. Each electrode includes nearly equivalent or equivalent surface areas for uniform current distribution. To prevent current from flowing, a nonconducting material is placed between the concentric circles. Current transfers from one electrode to another only when the user contacts the electrodes with their skin.

### FIELD OF THE APPLICATION

The present application relates to skin care and more particularly, to a system and method for applying both light and microcurrent to skin for treating and preventing acne, wrinkles, blemishes, fine lines, redness, stretch marks, sun spots, scars, and the like.

### BACKGROUND

Many conditions disfigure the skin. Acne vulgaris, one such condition, is formed by plugs within the infundibulum caused by cells, sebum, bacteria, and other debris. The plugs prevent cleansing of the sebum in the infundibulum and as a result of the continued production of sebum by the sebaceous gland, the infundibulum continues to stretch until either it or some lower portion of the follicle ruptures causing acne. In addition, other skin conditions disfigure the skin. Wrinkles are commonly the result of habitual facial expressions, aging, sun damage, smoking, poor hydration, and various other factors.

Several methods have been used for treating acne, wrinkles, and other skin conditions. One method includes applying a topical material to the skin. The user activates the topical material by applying energy to enlarge the pores, thereby treating the sebaceous gland. Other method includes applying chemical peels, mechanical abrasion and laser ablation. Through these other methods, the user sees cosmetic improvements by replacing old skin containing wrinkles with a new layer of horizontally oriented collagen in the superficial dermis.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one aspect of the present application, a system for treating and preventing skin conditions on a user is provided. The system includes a power source and a power management component for allocating power from the power source. The system includes a cathode and anode having a common center point, wherein the power management component supplies power to the cathode and anode when microcurrent therapy is desired. In addition, the system includes an insulator between the cathode and anode inhibiting electric charge from flowing between the cathode and anode. Current is capable of passing between the cathode and anode when both the cathode and anode contact the user. The system also includes a light source surrounding the cathode and anode, wherein the power management component supplies power to the light source when phototherapy is desired, the light source capable of directing light towards the user.

In accordance with another aspect of the present application, an apparatus for preventing and treating skin conditions is provided. The apparatus includes two or more electrodes, wherein the two or more electrodes are concentric circles. The apparatus also includes nonconducting material separating the two or more electrodes. The nonconducting material prevents current from passing between the two or more electrodes. When the two or more electrodes contact a user, however, current passes between the two or more electrodes through the user In addition, the apparatus includes an electromagnetic radiation source for providing light on the user.

In accordance with yet another aspect of the present application a skin care device is provided. The skin care device includes a power source, at least one processor, and memory operatively coupled to the processor. The memory stores program instructions that when executed by the processor causes the processor to perform at least one process selected from the group consisting of: i) allocate power from the power source to a circular cathode and anode having a common center point, whereby current passes from the cathode to the anode when the cathode and anode contact a user; ii) allocate power from the power source to an electromagnetic radiation source, the electromagnetic radiation directing energy on the user; and iii) a combination thereof.

In accordance with still yet another aspect of the present application, a kit is provided. The kit includes a base unit and an apparatus for treating and preventing user skin conditions and capable of recharging when coupled to the base unit. The apparatus includes two or more electrodes, wherein the two or more electrodes are concentric circles having a common center point and a gasket separating the two or more electrodes preventing current from passing between the two or more electrodes. When the two or more electrodes contact the user, current passes between the two or more electrodes. The apparatus also includes an electromagnetic radiation source.

In accordance with another aspect of the present application, a method for treating skin conditions is provided. The method includes determining at least one type of treatment selected by a user. The method applies a current to the user through two or more electrodes if the selected treatment includes microcurrent therapy. The two or more electrodes are concentric circles separated by a nonconducting material that prevents current from passing between the two or more electrodes. Current passes between the two or more electrodes when the two or more electrodes contact the user. The method also applies a light source on the user if the selected treatment includes photorejuvenation therapy.

In accordance with a yet another aspect of the present application, a method for treating and preventing skin conditions on a user is provided. The method includes selecting at least one type of therapy and placing two or more electrodes on a target area if the user selected microcurrent therapy. The two or more electrodes are concentric circles with current passing between the two or more electrodes through the user when the two or more electrodes contact the user. The method also includes directing a light to the target area if the user selected phototherapy.

In accordance with another aspect of the present application, a device for skin care using both microcurrent and light is provided. The device includes a power source and a user interface for determining user selections. In addition, the device includes two or more electrodes, wherein the two or more electrodes are concentric circles. Power is supplied to the two or more electrodes from the power source when the user selects microcurrent therapy from the user interface. The device also includes insulating material separating the two or more electrodes preventing current from passing between the two or more electrodes. Current passes between the two or more electrodes when the two or more electrodes contact the user. Furthermore, the device includes an electromagnetic radiation source, power being supplied to the electromagnetic radiation source from the power source when the user selects phototherapy from the user interface.

In accordance with yet another aspect of the present application, a user skin care device is provided. The device includes a microcurrent therapy component having two or more circular electrodes with a common center. The microcurrent therapy component is capable of passing current between the two or more electrodes when the two or more electrodes contact the user. The device also includes an electromagnetic radiation therapy component having a light source and capable of directing light towards the user.

In accordance with another aspect of the present application, an apparatus for treating and preventing skin conditions on a user is presented. The apparatus includes a power source and a power management component for allocating power from the power source. The apparatus also includes a user interface and a cathode and anode. The cathode and anode are concentric circles having a common center. The power management component supplies power to the cathode and anode when microcurrent therapy is selected from the user interface.

The apparatus includes a nonconducting material between the cathode and anode, which prevents electric charge from flowing between the cathode and anode. The current is capable of passing between the cathode and anode through the user when both the cathode and anode contact the user. The cathode and anode have nearly equivalent or equivalent surface areas producing even current flow.

In addition, the apparatus includes a light source surrounding the cathode and anode. The power management component supplies power to the light source when phototherapy is selected from the user interface. The light source is capable of directing light towards the user. The light is made from a plurality of LEDs producing a wavelength of about 630 nanometers.

### BRIEF DESCRIPTION OF DRAWINGS

The novel features believed to be characteristic of the application are set forth in the appended claims. In the descriptions that follow, like parts are marked throughout the specification and drawings with the same numerals, respectively. The drawing figures are not necessarily drawn to scale and certain figures may be shown in exaggerated or generalized form in the interest of clarity and conciseness. The application itself, however, as well as a preferred mode of use, further objectives and advantages thereof, will be best understood by reference to the following detailed description of illustrative embodiments when read in conjunction with the accompanying drawings, wherein:

FIG. 1 is a diagram representing illustrative layers and structures contained within skin in accordance with one aspect of the present application;

FIG. 2 is a diagram showing an exemplary skin care device in accordance with one aspect of the present application;

FIG. 3 is an illustration showing a sample kit for retaining the skin care device in accordance with one aspect of the present application;

FIG. 4 is a block diagram showing exemplary components of the skin care device in accordance with one aspect of the present application;

FIG. 5 is a schematic illustrating components of the exemplary skin care device in accordance with one aspect of the present application;

FIG. 6A is a diagram showing current flowing from an inner cathode to an outer anode on an exemplary skin care device in accordance with one aspect of the present application;

FIG. 6B is a diagram showing current flowing from an outer cathode to an inner anode on an exemplary skin care device in accordance with one aspect of the present application;

FIG. 7A is a diagram showing a line whereby a cross-section of the exemplary skin care device is taken in accordance with one aspect of the present application;

FIG. 7B is an illustrative cross-section of the exemplary skin care device in accordance with one aspect of the present application;

FIG. 8 is a diagram showing an exemplary medium or surface for circulating current in accordance with one aspect of the present application;

FIG. 9A is a graph depicting voltages applied by the skin care device using 500 ohms of resistance in accordance with one aspect of the present application;

FIG. 9B is a graph depicting voltages applied by the skin care device using 2K ohms of resistance in accordance with one aspect of the present application;

FIG. 9C is a graph depicting voltages applied by the skin care device using 10K ohms of resistance in accordance with one aspect of the present application;

FIG. 9D is a graph depicting pulse groups applied by the skin care device using a 500 ohm resistor in accordance with one aspect of the present application; and

FIG. 10 is a flow chart illustrating an exemplary method for treating and preventing skin conditions in accordance with one aspect of the present application.

### DETAILED DESCRIPTION

The detailed description set forth below in connection with the appended drawings is intended as a description of presently-preferred embodiments of the application and is not intended to represent the only forms in which the present application may be constructed and/or utilized. The description sets forth the functions and the sequence of steps for constructing and operating the application in connection with the illustrated embodiments. It is to be understood, however, that the same or equivalent functions and sequences may be accomplished by different embodiments that are also intended to be encompassed within the spirit and scope of this application.

Generally described, the present application relates to a system and method for skin care. In particular, the present application relates to systems and methods for treating and preventing skin conditions by using microcurrents, electromagnetic radiation, or a combination thereof. In an illustrative embodiment, the device has two or more electrodes. The electrodes are concentric circles surrounding a common center. Typically, nonconducting material separates the two or more electrodes. The nonconducting material prevents current from passing between the two or more electrodes. Current, however, passes between the two or more electrodes and around the nonconducting material when the two or more electrodes contact the user. In addition, the device includes an electromagnetic radiation source. In typical embodiments, the electromagnetic radiation source applies visible light to the user. Through the combination of microcurrent and electromagnetic radiation, the device presented in this application provides a valuable tool for preventing and treating skin conditions.

It should be understood that the following description is presently largely in terms of treatment for skin conditions. One skilled in the relevant art, however, will appreciate that the disclosed embodiments are illustrative in nature and should not be construed as limiting. As such, the present application should be construed as providing measures to prevent future skin conditions as well. Furthermore, the description presented herein is not limited to treatment of skin on a user's face, but instead includes any type of tissue where the beneficial aspects of applying microcurrents and electromagnetic radiation can be realized.

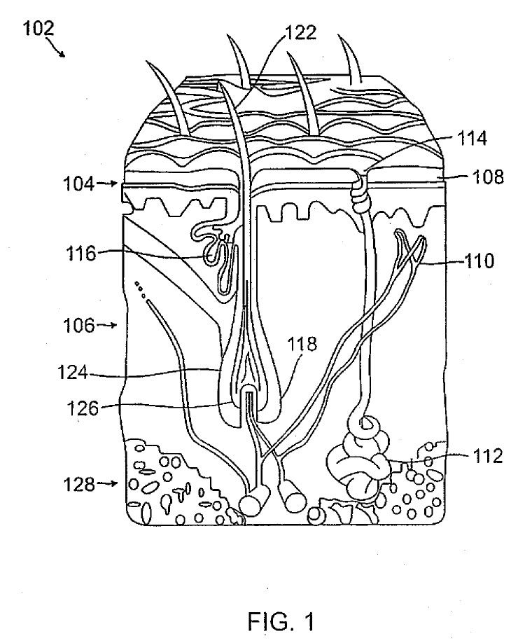

Skin, the outer covering of living tissue of an animal, provides many important functions. Skin protects animals from pathogens entering into the body. Skin also provides insulation, sensation, synthesis of vitamin D, protection of vitamin B folates, and regulates temperatures. As depicted in FIG. 1, skin 102 is composed of three primary layers: the epidermis 104, the dermis 106, and the hypodermis 120. The epidermis 104 waterproofs and serves as a barrier to infection, while the dermis 106 serves as a location for the appendages of skin 102. The hypodermis or subcutaneous tissue 120 is used for fat storage.

The epidermis 104, the outer most layer of the skin 102, contains no blood vessels, but its deepest layer is supplied with lymph fluid. Typically, the epidermis 104 is thickest on the palms and feet of an animal. The surface layer of the epidermis 104 may contain twenty-five to thirty sub layers of flattened scale-like cells. Cells are continuously cast off by friction and replaced by cells from the deeper epidermal 104 layers. These cells are commonly called keratinized cells because the living matter inside the cell is changed to a protein that helps to give the skin 102 its protective properties.

New cells are often formed in the deepest layer within the epidermis 104, called the stratum corneum 108. New cells gradually move towards the outer layers of the skin 102 as the stratum corneum 108 is abraded or shed. New cells change in form as they move upward to the outer layers becoming keratinized in the process.

The dermis 106 is the tough elastic layer containing white fibrous tissue interlaced with yellow elastic fibers. Located within the dermis layer 106 are blood vessels 110, lymphatic capillaries and vessels, sweat glands 112 and their ducts 114, sebaceous glands 116, sensory nerve endings, arrectores pilorum, hair follicles 122, hair bulbs 118, and hair roots that have an outer root sheath 124 and inner root sheath 126.

Hypodermis 120, the deepest of the layers of skin 102, is located on the bottom and connects or binds the dermis 106 above it to the underlying organs. The hypodermis 120 layer is composed of loose fibrous connective tissue and fat (adipose) cells interlaced with blood vessels. In females, the hypodermis 120 is generally about 8% thicker than in males. The hypodermis 120 provides insulation, stores lipids, cushions the body, and regulates temperatures.

Dead cells continually accumulate with secretions of sweat and dust forming a filthy layer on the surface of skin 102. The filthy layer decomposes and produces bacterial flora. The skin 102 becomes disturbed when it is excessively dirty making it more susceptible to damage.

Oily skin 102 also causes damage to skin 102. Overactive glands 116 produce a substance called sebum, a naturally health skin 102 lubricant. When skin 102 produces excessive sebum, it becomes heavy and thick in texture. Oily skin 102 is characterized by shininess, blemishes, and pimples. Oily skin 102 is susceptible to clogged pores, blackheads, and buildup of dead skin 102 cells on the surface of skin 102. Severely damaged skin 102 tries to heal by forming scar tissue. This is often discolored and depigmented.

Aging skin 102, another concern, causes skin 102 to become thinner and more easily damaged. Intensifying this effect is the decreasing ability of skin 102 to heal itself. Skin 102 aging is caused by the fall in elasticity because of less blood flow and lower gland activity.

Skin 102, subjected to constant attack, can be afflicted with numerous ailments. Tumors, skin 102 cancer, rashes, blisters, acne, keratosis pilaris, fungal infections, microbial infections, calcinosis cutis, sunburn, keloid, scabies, vitiligo, albinism, eczema, and psoriasis are ailments that can affect skin 102 and the way it looks.

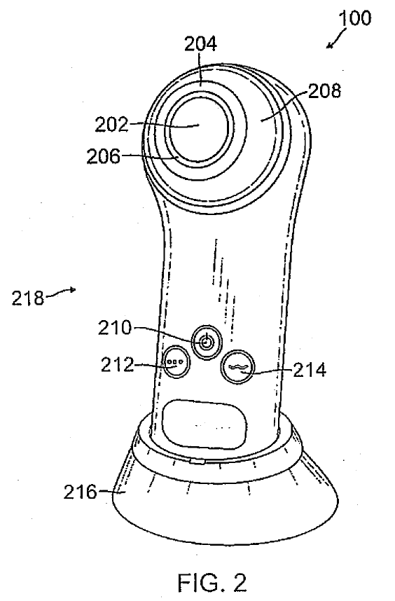

To treat and prevent skin 102 conditions, device 100 combines microcurrent therapy and photorejuvenation (or phototherapy) as depicted in FIG. 2. Device 100 firms and lifts skin 102 and in addition, refines the tone and texture of skin 102. Device 100 also reduces fine lines and wrinkles. While effects of photorejuvenation therapy generally occur after several days, results of microcurrent therapy occur almost immediately.

Surrounding device 100 is enclosure unit 216. The handheld enclosure unit 216 provides an easy to clean surface and comes in multiple shapes and sizes. Enclosure unit 216 protects internal components and includes multiple external components. The external components are positioned in easy to find locations in order for the user to operate device 100 without difficulty.

Preferably, enclosure unit 216 is made from plastic. Plastic is easy to clean and durable. Alternatively, enclosure unit 216 can be made of metal, composite, or the like. Enclosure unit 216 may also include gripping material so the user can handle device 100 without difficulty.

The weight and dimensions of device 100 and enclosure unit 218 vary dependent on components located within device 100. Alternatively, the weight and dimensions of device 100 and enclosure unit 218 do not have to conform to the internal components of the device 100 and may take any desired or preferred shape. Typically, however, device 100 will be 6 inches by 1.75 inches by 2.25 inches with a weight of 4.7 ounces with the power source included.

While the human body often produces electrical currents to repair itself, often this is not enough. Device 100 incorporates a microcurrent therapy component to produce additional electrical current. Before describing specific features, however, a discussion on beneficial effects of microcurrents will be presented.

Generally, microcurrents relieve pain and heal tissue by affecting the injured tissue at the cellular level. Muscle, tendon, bone, nerve, and skin 102 are tissues that have responded positively to microcurrent therapy. Microcurrent therapy neutralizes the oscillating polarity of injured cells, increases availability of adenosine triphosphate, and increases cell permeability. Microcurrents also increase local protein synthesis.

By moving ions and larger charged molecules in the blood and tissue, microcurrents cause movement of fluid within the body and tissue. Larger currents are useful for fast, short-term effects, whereas smaller currents (in the hundreds of microamps) are more useful for long-term effects.

Nocioceptive fibers, the free nerve endings sensitive to tissue dysfunction, are found in variety of tissues including skin 102, fibrous capsule periosteum intramuscular arteries, and blood vessel walls. Deformation of nocioceptive fibers may cause stimulation and therefore reporting of pain to the brain. In order for this pain to be reduced, microcurrents can be applied to the fibers. Microcurrents work by decreasing pain and reducing the cause of pain by altering electrical activity surrounding the injured area. Microcurrents are effective on areas of increased blood supply and increased nocioceptive fibers.

Microcurrents produced by device 100 can vary, but typically are very small. In preferred embodiments, microcurrents produced by device 100 vary from about 2 microamps up to about 35 microamps with a deviation of plus or minus 5 percent. Alternatively, device 100 can produce microcurrents less than 600 microamps. Various modes of application, adjustable treatment variables, and relatively few contraindications characterize device 100.

Electricity will generally take the path of least resistance. As such, traditional electrical charges placed on the body travel around traumatized cells. A smaller current, one that can separate the cell and balance the cell electrically, can restore a more normal physiological state to the damaged cells. Small electrical charges may be helpful in initiating and perpetuating the numerous electrical chemical reactions in the healing process.

Waveforms used are typically a positive direct current, negative direct current or a combination of these in which the polarity is switched at an adjustable rate (usually 0.3 hertz to 30 hertz using a 50 percent duty cycle waveform). Preferably, device 100 adjusts at about 8.7 hertz. The frequency of the current may be adjusted dependent on the user's type of skin 102 tissue. The use of microamps in microcurrent therapy, results in little or no patient discomfort or sensation during application. Alternating or pulsed electrical currents can be used to drive, stimulate, and promote healing of tissue.

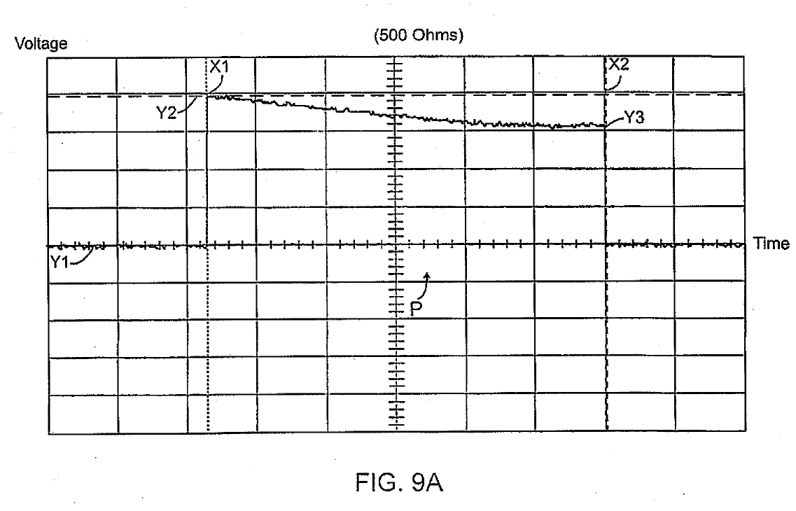

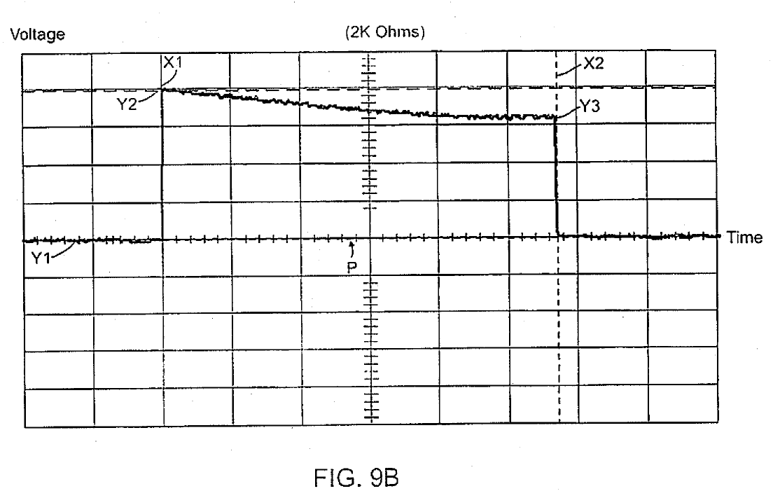

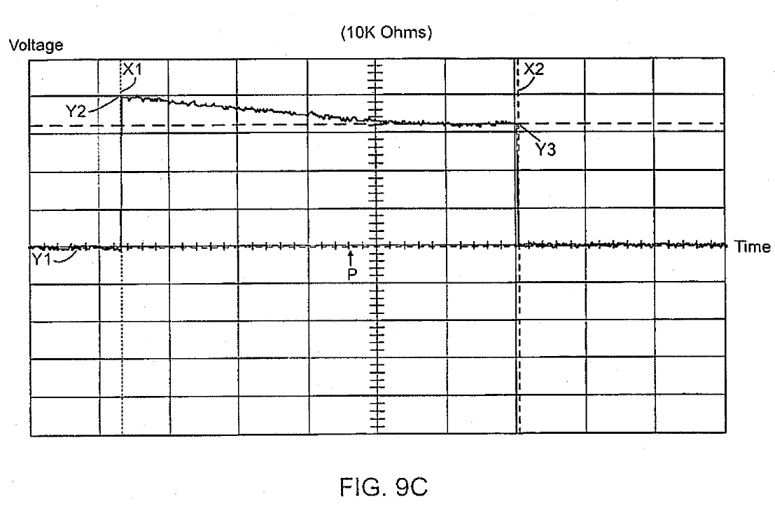

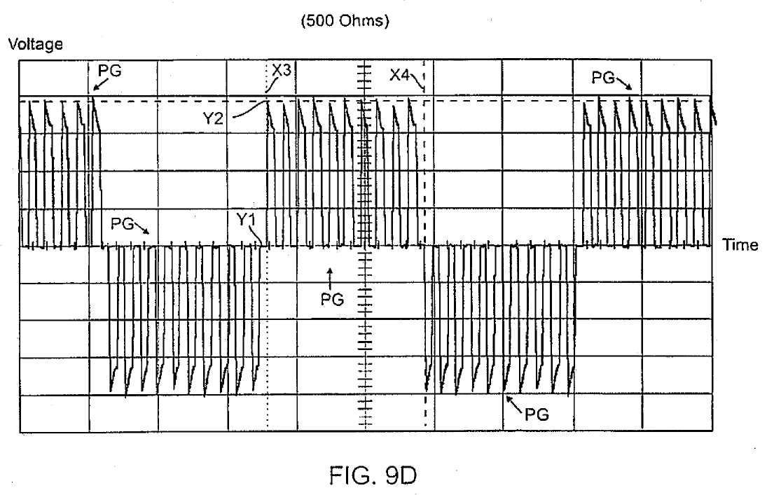

Device 100 uses monophasic waveforms having rectangular shapes. Pulse widths are about 58 milliseconds, while pulse groups are about 1.15 seconds. Typically, pulses produced by device 100 are on for about 57 milliseconds and off for about 58 milliseconds giving rise to the graph depicted in FIG. 9D. While exemplary characteristics of device 100 have been presented, device 100 provides alternative waveforms, pulse widths, and pulse durations.

In typical treatments, microcurrents are initially used to reduce pain. This initial mode, also called the pain mode, consists of a short ramp of about 0.01 seconds, a frequency of about 30 hertz, and a current of about 80 to 100 microamps. The pain mode is generally followed by a healing mode. The healing mode consists of a longer ramp of about 2 seconds, a frequency of about 0.3 hertz, and a current of about 20 to 40 microamps. Average treatments include about 10 minutes on the pain mode followed by about 10 to 20 minutes on the healing mode. Treatment should be performed every other day, or daily for optimal results. In some embodiments, device 100 may provide patients with the benefit of a combined therapy using a pain and healing mode. The availability and use of different electrotherapy methods is often critical to treatment immediately post injury.

With reference to the illustration shown in FIG. 2, microcurrent therapy component incorporates two or more electrodes 202 and 204. Electrodes 202 and 204 are placed so that an electrical pathway can be followed within the user. Generally, the pathway follows muscular electrical flow, down a muscular pathway of radiating nerve pain, through trigger points, or medial/lateral through a swollen joint.

While adhesive material for electrodes 202 and 204 can be applied, it is not required. Without using adhesives, device 100 reduces irritation caused by those adhesives, which is typical of other skin 102 prevention and treatment devices.

Distribution of current between electrodes 202 and 204 is important and thus, placement of the electrodes 202 and 204 is critical to effective treatment. Because patients often lack the anatomical knowledge needed to effectively place electrodes 202 and 204 by themselves, frequent clinic visits and limitations on therapeutic activities are made. To overcome these deficiencies, device 100 sets the placement of electrodes 202 and 204 therefore reduces the need for clinic visits.

Electrodes 202 and 204 can be made from several different materials. However, in a preferred embodiment of device 100, electrodes 202 and 204 are made of metal, alloy, or combination thereof in order to conduct electricity.

When charged, one electrode 202 and 204 will be a cathode and the other electrode 202 and 204 an anode. Anodes are electrodes 202 and 204 that loose electrons. Cathodes are electrodes 202 and 204 that gain electrons from the anodes. Each electrode 202 and 204 may become either the anode or cathode depending on the voltage applied to electrodes 202 and 204. Furthermore, the voltage between electrodes 202 and 204 may be regulated.

Direct current typically flows between electrodes 202 and 204. Nonetheless, electrodes 202 and 204 can have alternating current. In embodiments having alternating current neither electrode 202 and 204 is designated anode or cathode since the direction of flow of the electrons changes periodically, usually many times per second.

While preferred embodiments incorporate two electrodes 202 and 204, multiple electrodes 202 and 204 may be used. When multiple electrodes 202 and 204 are used, cathodes and anodes may alternate between each other. Alternatively, electrodes 202 and 204 may incorporate multiple anodes positioned in the center surrounded by multiple cathodes. Contrawise, multiple cathodes may surround multiple anodes.

Preferably, electrodes 202 and 204 are concentric circles having a common center point. Through concentric electrodes 202 and 204, current flows evenly. Furthermore, the surface area of the electrodes 202 and 204 are nearly equivalent or equivalent so that current uniformly flows between the electrodes 202 and 204. These features will be described in more detail below.

In some instances, electrical current passes through air from one electrode 202 and 204 to the other and not through the intended surface or medium. As such, separating the electrodes 202 and 204 with air results in the loss of electric current. To prevent this, nonconducting material 206 separates electrodes 202 and 204.

Nonconducting material 206 resists the flow of electrical current and prevents current from passing between electrodes 202 and 204 when charged. Typically, nonconducting material 206 lacks mobile charges, the nonconducting material 206 having atoms with tightly bounded valence electrons. Preferably, nonconducting material 206 is made of silicone, but Teflon is also a very good electrical nonconducting material 206. Rubber-like polymers and most plastics are also good nonconducting materials 206.

While the terms “nonconducting material 206” are described and used throughout this application, other terms such as insulators, dielectric materials, gasket, and the like may be interchanged with “nonconducting material 206”.

In some instances, nonconducting material 206 generates heat. Heat typically appears as a result of preventing the flow of electrons between electrodes 202 and 204. In general, all materials offer some resistance and warm up when current flows.

To dispel heat generated from nonconducting material 206, device 100 can include cooling components. Such components remove heat and can be placed internally or externally on the device 100. Device 100 may also shut down for brief intervals allowing heat to dissipate from the nonconducting material 206.

Nonconducting material 206, separating electrodes 202 and 204, may be shaped as a concentric circle having the same center as electrodes 202 and 204. Nonconducting material 206 prevents current from passing between electrodes 202 and 204 without another surface or medium to travel through i.e. a user. The width of nonconducting material 206 varies dependent on the desired distance set by the user. Generally speaking, the larger the width, the further the current travels. On the other hand, the current travels very little when the width of nonconducting material 206 is smaller.

Device 100 also incorporates an electromagnetic radiation source or component 208 for additional therapy. The electromagnetic radiation component 208 can transmit radio waves, microwaves, terahertz radiation, infrared radiation, visible light, ultraviolet radiation, X-rays and gamma rays. Preferably, however, electromagnetic radiation component 208 is a light source and provides photorejuvenation (or phototherapy) to the user.

In applications of light to skin 102, the light typically activates porphyrin in propionibacterium acnes which damages and ultimately kills bacteria by releasing singlet oxygen. The light also activates adenosine triphosphate in the skin 102, the adenosine triphosphate maintaining cell structure and shortening actin and myosin filament crossbridges required for muscle contraction.

Electromagnetic radiation component 208 can provide light having varying wavelengths, each wavelength providing multiple beneficial aspects. For instance, yellow light having wavelengths from about 530 nanometers to about 600 nanometers applied to skin 102 treats rosacea and sunspots. Red light having wavelengths from about 630 nanometers to about 680 nanometers applied to skin 102 treats fine lines, wrinkles, lesions, canker sores, and inflammation. Green light having wavelengths from about 500 nanometers to about 530 nanometers applied to skin 102 treats hyperpigmentation, brown spots, and age spots. Blue light having wavelengths ranging from about 400 nanometers to about 470 nanometers applied to skin 102 kills bacteria that cause acne. Infrared light having wavelengths from about 800 nanometers to about 1000 nanometers applied to skin 102 treats inflammation, lesions, and canker sores.

As depicted in FIG. 2, electromagnetic radiation component 208 surrounds electrodes 202 and 204. Preferably, electromagnetic radiation component 208 shares the same common center point of electrodes 202 and 204. In other embodiments, electromagnetic radiation component 208 incorporates four separate LEDs positioned around electrodes 202 and 204. Typically, electromagnetic radiation component 208 is on the outer perimeter of the head of device 100. However, other embodiments may incorporate the electromagnetic radiation component 208 inside the concentric circles created by electrodes 202 and 204 or electrode 204. In addition, nonconductive material 206 may also be placed between the electromagnetic radiation source 208 and electrodes 202 and 204.

Each described therapy presented above can be delivered through handheld enclosure unit 218. Enclosure unit 218 houses components of device 100 and a user interface for providing therapy selections. In preferred embodiments, the user interface incorporates three buttons 210, 212, and 214. Button 210 turns on and off the device 100. Button 212 operates the photorejuvenation therapy mode and button 214 controls the photorejuvenation therapy mode.

Individual therapies or a combination of both can be selected depending on the user. Importantly, and because each treatment typically works at different rates from the other, buttons 212 and 214 allow for separate treatments at different times. Each button 210, 212, and 214 can include an LED under the button 210, 212, and 214 that lights up when it has been selected by the user.

The user interface of device 100 can also incorporate controls for selecting the intensity of each treatment. For example, the intensity of microcurrents passing between the electrodes 202 and 204 can be adjusted. Alternating current or direct current between the electrodes 202 and 204 can also be selected. Separate controls can vary the electromagnetic radiation source 208. A low battery indicator can be placed on the interface.

Handheld enclosure unit 218 can be completely detached from base 216. In one embodiment, base 216 recharges the battery of device 100. In this embodiment, a cord attached to the base 216 extends therefrom and into a suitable outlet. Typically, the electrical cords plugs into an outlet providing 110-120 volts of electricity. However, the cord may have numerous shapes and sizes depending on the standards provided by each country in which device 100 is used.

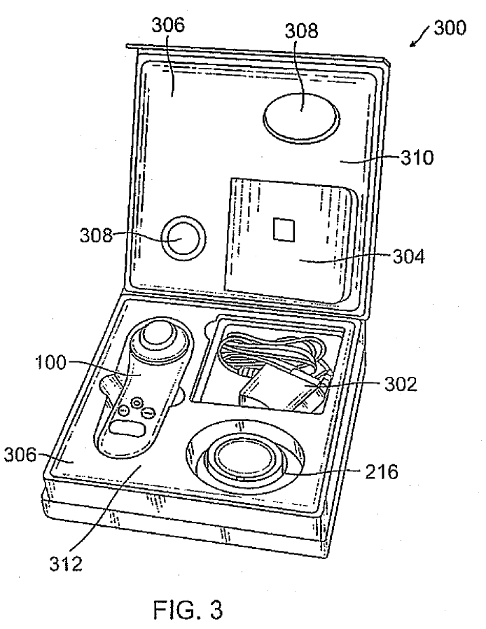

As shown in FIG. 3, components of device 100 can be stored and maintained using kit 300. Kit 300 contains a foldable first half 310 and second half 312. Each half 310 and 312 maintains multiple cushions 306, the cushions 306 having many apertures 308 for storing components of device 100.

Enclosed within kit 300 is device 100 along with base unit 216. In addition, kit 300 includes the previously mentioned cord 302. In some instances, kit 300 may include topical cream 304. Topical cream 304 can repair deep tissue. By applying cream 304 to a user's face and pressing cream 304 into the user's skin 102, topical cream 304 allows greater penetration of the therapies provided by device 100.

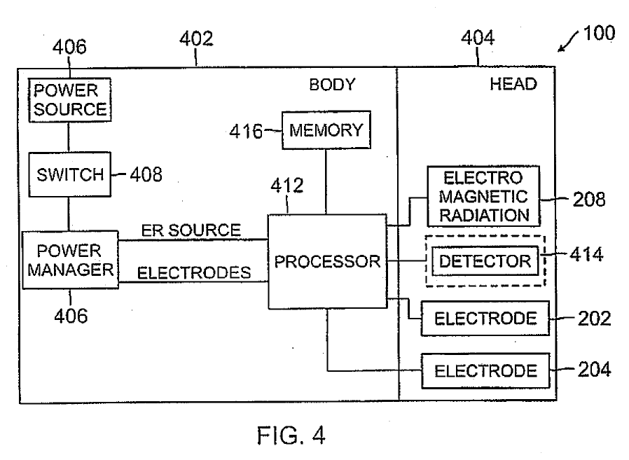

Device 100 includes multiple components as depicted in FIG. 4. Device 100 contains a body portion 402 and a head portion 404. The body portion 402 incorporates the majority of the device 100 and provides housing for power source 406, switch 408, power manager 410, processor 412, and memory 416.

Power source 406 provided in the body portion 402 of the device 100 is typically a battery 406. Batteries 406 allow device 100 to be portable and separate from base 216. Two types of batteries 406 can be used with device 100: a primary battery 406 and a secondary battery 406. The batteries 406 generally produce 5 volts.

Primary batteries 406 transform chemical energy to electrical energy. When the initial supply of reactants is exhausted, energy cannot be readily restored to the battery 406 by electrical means. Secondary batteries 406 are capable of being recharged. These batteries 406 have their chemical reactions reversed by supplying electrical energy to the cell. By applying device 100 to base 216, device 100 can be recharged for further use. After recharging, secondary batteries 406 are often restored to their original composition.

While losing its portability features, device 100 may include a power source 406 that is directly attached to an outlet. Power from the outlet may be converted so that device 100 can operate.

Coupled to power source 406 is switch 408. Switch 408 corresponds to button 210. When button 210 is pressed, switch 408 closes the circuit, the closed circuit providing power from power source 406 to power management component 410. Power management component 408 provides power to processor 412 from the power source 406 as well as stores power. Processor 412 distributes power to the electromagnetic radiation component 208, electrodes 202 and 204, or both based on a user's selection.

Processor 412 coupled to memory 416 processes selections made by the user though buttons 212 and 214. Recall that button 212 activates photorejuvenation therapy mode, while button 214 activates microcurrent therapy mode. With the press of each button 212 and 214, processor 412 retrieves instructions from memory 416 and executes the instructions according to the treatment selected. When both buttons 212 and 214 are selected, both the instructions for photorejuvenation and microcurrent therapy will be retrieved and executed. In a multi-processor system, both instructions can be retrieved and executed simultaneously.

Processor 412 may execute instructions causing delays so that the user may properly apply the device 100 to a target area. By executing instructions from memory 416, processor 412 allocates power from the power source 406 to electrodes 202 and 204, electromagnetic radiation source 208, or a combination thereof.

Head 404 of device 100 receives power allocated by processor 412 and directs that power to the appropriate destination. While head 404 in preferred embodiments is stationary, head 404 can also pivot so that the energy produced by the device 100 can be directed to a target area without moving the body 402 of device 100.

Optionally, head 404 also contains detector 414. Detector 414, coupled to processor 412, saves energy by determining whether a user has placed head 404 against a surface i.e. the user's body or face. When the user's body or face is detected, processor 412 executes instructions allocating power to the appropriate destination. On the other hand, no power is allocated when the user's body or face is not detected.

Possibilities for implementing detector 414 incorporate the use of electrodes 202 and 204 to determine whether the user's body or face is present. In other implementations, switches on top of head 404 are closed when the user presses their body or face against head 404. Numerous possibilities exist for detecting a user's body or face.

Detector 414 also monitors temperatures of head 404. When components such as the electrodes 202 and 204 or the electromagnetic radiation component 208 dispel large amounts of energy and become hot past a certain threshold, detector 414 sends a signal to processor 412 to intermittently shut device 100 down. Such threshold may occur when contact with the head 404 against the user's body or face burns the user.

Furthermore, device 100 automatically shuts down after a predetermine time has passed. Typically, device 100 shuts down after 5 minutes. Some embodiments, will allow users to override the shut off command.

Generally, components located in head 404 can be easily produced making head 404 easily interchangeable. Through simple connectors, head 404 can snap on and off body 402. Many benefits can be realized by interchanging head 404. One benefit includes a clean surface on which treatment may be performed. Because of the amount of dirt and other harmful substances that can be found on the head 404 after several uses, the user should replace head 404 often.

Alternatively, user's can place plastic over the head 404 itself. The plastic cover maintains both benefits of using microcurrents and electromagnetic radiation as well as providing a new surface for the user after each use.

Head 404 can also be interchanged to provide different therapies or treatments. For example, electromagnetic radiation component 208 can be interchanged to provide radio waves, microwaves, terahertz radiation, infrared radiation, visible light, ultraviolet radiation, X-rays and gamma rays. In addition, head 404 can be interchanged to provide electrodes 202 and 204 with larger or smaller surface areas. By having a smaller surface area, users can direct treatment to a specific area. Having a larger surface area, on the other hand, allows for large areas of treatment.

Schematically, components of device 100 are illustrated in FIG. 5. Body 402 of device 100 includes battery 406, switch 408, power management component 410, and processor 412. Battery 406 supplies power to the circuit when switch 408 is closed. Power management component 410 distributes power to processor 412. Dependent on selections made by the user, processor 412 distributes the power to the electromagnetic radiation component 208, electrodes 202 and 204, or both.

Attached to processor 412 are lines 502, 504, 506, and 508 that pass from body 402 to head 404 of device 100. Line 502 couples processor 412 to electrode 202, while line 504 couples processor 412 to electrode 204. Through lines 502 and 504 current may pass from one electrode 202 and 204 to another.

In preferred embodiments, and as depicted in FIG. 5, electrodes 202 and 204 are concentric circles have a common center point. Radially extending lines from the common center linking electrodes 202 and 204 provide the shortest distance between electrodes 202 and 204. Because current tends to typically flow through the shortest distance, device 100 provides for even current flow as the distances between electrodes 202 and 204 are uniform.

In other embodiments, other shaped electrodes 202 and 204 may be used. However, these shapes do not provide the uniform distances as discussed above, and as a result, current generally will not flow evenly.

In preferred embodiments, the surface area of electrode 202 is equivalent or nearly equivalent to the surface area of electrode 204. When the surface areas are equivalent, electrons escaping from the anode typically match those received by the cathode. Given that the surface areas of electrodes 202 and 204 are nearly equivalent or equivalent, electric current is distributed uniformly or evenly from one electrode 202 and 204 to another. In alternative embodiments, electrodes 202 and 204 may have different surface areas.

In addition, the surface of electrodes 202 and 204 can be made quite large. Generally, the greater the surface area of electrodes 202 and 204, the more electrons capable of escaping the anode and being received at the cathode at any given time.

Current typically travels the opposite direction of electron flow. As such, current travels from the cathode to the anode, while electrons flow from the anode to the cathode. Dependent on the type of electrodes 202 and 204 used, positive charges, instead of negative charges, may flow between electrodes 202 and 204. In these instances, current flows in the direction of the positive charges.

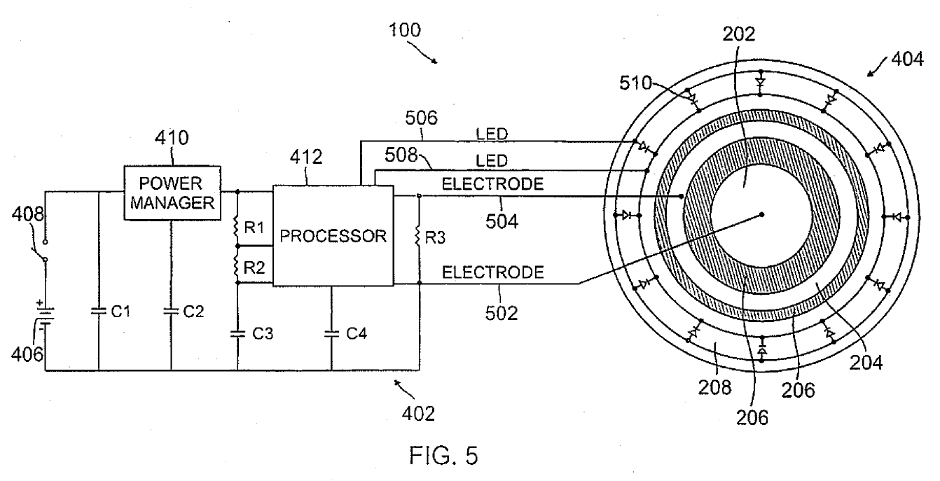

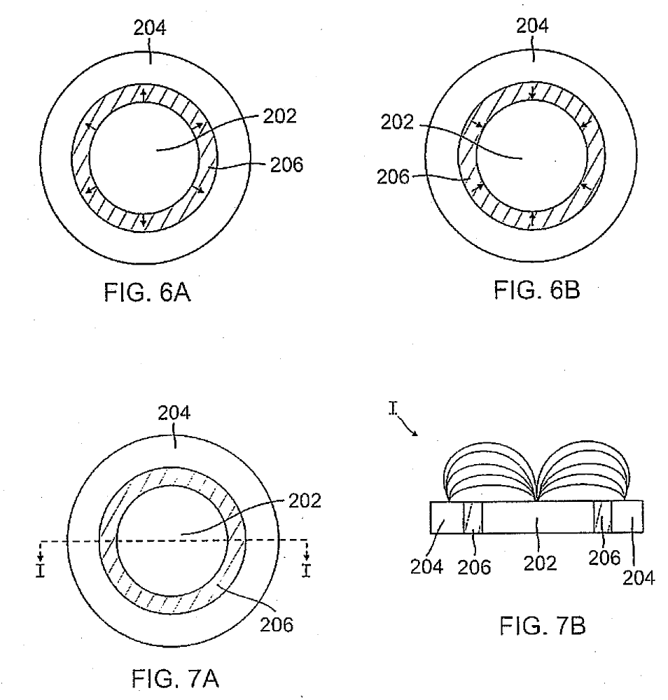

When electrode 202 is the cathode and electrode 204 is the anode, current flows from the inner electrode 202 to the outer electrode 204 as depicted in FIG. 6A. Electron flow travels in the opposite direction i.e. from the outer electrode 204 to the inner electrode 202. Conversely, current flows from the outer electrode 204 to the inner electrode 202 when electrode 204 is a cathode and electrode 202 is an anode as depicted in FIG. 6B. In this embodiment, electrons flow from electrode 202 to electrode 204.

While FIGS. 6A and 6B describe current traveling in a single direction, device 100 may also alternate current between the two electrodes 202 and 204. As such, electrode 202 can become a cathode and an anode, and alternatively, electrode 204 can become an anode and cathode.

As recited above, nonconducting material 206 prevents the flow of electrons. Because the nonconducting material 206 prevents electrons from flowing, another medium is required for electrons to flow between electrodes 202 and 206.

To dissect how device 100 uses microcurrents to prevent and treat skin 102 conditions, cross section I is taken as shown in FIG. 7A. Cross section I encompasses electrodes 202 and 204 and nonconducting material 206. When the two or more electrodes 202 and 204 contact the user, current passes between electrodes 202 and 204 through the user and around nonconducting material 206 as depicted in FIG. 7B. Current can travel between electrodes 202 and 204 through curved lines from all directions, except through nonconducting material 206.

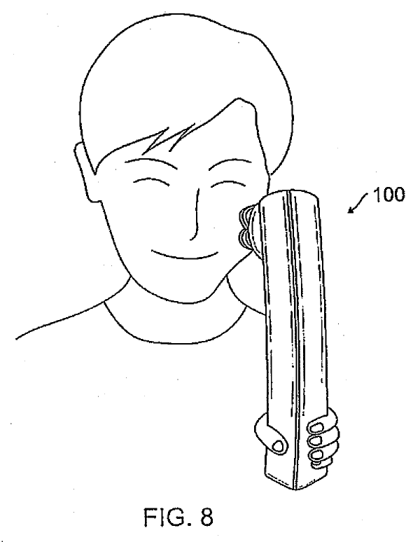

Typically, surfaces that are capable of passing current between electrodes 202 and 204 and nonconducting material 206 have electrons that move freely. Other surfaces may include a user's body, and more particularly, a user's face as depicted in FIG. 8.

Performance of device 100 is based on multiple criteria. Maximum output voltages of electrodes 202 and 204 are about 17.7 Vdc at 500 ohms, about 18.0 Vdc at 2K ohms, and about 17.7 Vdc at 10K ohms. The maximum output current is about 35 microamps at 500 ohms, about 9 microamps at 2K ohms, and about 2 microamps at 10K ohms. For device 100, the net charge is about 2018 microcoulombs at 500 ohms, while the maximum current density is about 0.008 microamps per centimeter squared at 500 ohms. The maximum power density is about 0.0003 watts per centimeter squared at 500 ohms.

Graphically, FIG. 9A represents generated voltages of pulse P between electrodes 202 and 204 with a resistance of 500 ohms. The Y-Axis represents voltages and each division represents 5 volts. The X-Axis represents time and each division represents 5 milliseconds. Pulse P is the difference between time X2 and X1, which in this graph is about 57 milliseconds.

Before time X1, the voltage between electrodes 202 and 204 is at Y1, which is about 0 volts. At time X1, voltage between electrodes 202 and 204 is at Y2, the maximum potential between the electrodes 202 and 204. At 500 ohms, voltage Y2 is about 19.7 Vdc. From there, the voltage between electrodes 202 and 204 begins to decrease until time X2. At time X2, the voltage between electrodes 202 and 204 is at Y3, which is about 15.7 Vdc, the lowest point of pulse P. Thereafter, voltages for times greater than X2 are at Y1, which is about 0 volts. The voltage average for pulse P is about 17.7 Vdc.

FIG. 9B represents voltages of pulse P between electrodes 202 and 204 with a resistance of 2K ohms. The Y-Axis represents voltages and each division represents 5 volts. The X-Axis represents time and each division represents 5 milliseconds. Pulse P is the difference between time X2 and X1, which in this graph is about 57 milliseconds.

Before time X1, the voltage between electrodes 202 and 204 is at Y1, which is about 0 volts. At time X1, voltage between electrodes 202 and 204 is at Y2, the maximum potential between the electrodes 202 and 204. At 2K ohms, voltage Y2 is about 19.9 Vdc. From there, the voltage between electrodes 202 and 204 begins to decrease until time X2. At time X2, the voltage between electrodes 202 and 204 is at Y3, which is about 16.0 Vdc, the lowest point of pulse P. Thereafter, voltages for times greater than X2 are at Y1, which is about 0 volts. The voltage average for pulse P is about 18.0 Vdc.

FIG. 9C represents voltages of pulse P between electrodes 202 and 204 with a resistance of 10K ohms. The Y-Axis represents voltages and each division represents 5 volts. The X-Axis represents time and each division represents 5 milliseconds. Pulse P is the difference between time X2 and X1, which in this graph is about 58 milliseconds.

Before time X1, the voltage between electrodes 202 and 204 is at Y1, which is about 0 volts. At time X1, voltage between electrodes 202 and 204 is at Y2, the maximum potential between the electrodes 202 and 204. At 10K ohms, voltage Y2 is about 19.7 Vdc. From there, the voltage between electrodes 202 and 204 begins to decrease until time X2. At time X2, the voltage between electrodes 202 and 204 is at Y3, which is about 15.6 Vdc, the lowest point of pulse P. Thereafter, voltages for times greater than X2 are at Y1, which is about 0 volts. The voltage average for pulse P is about 17.7 Vdc.

FIG. 9D is a graph depicting pulse groups PG between electrodes 202 and 204 applied by device 100 using a 500 ohm resistor. The Y-Axis represents voltages and each division represents 5 volts. The X-Axis represents time and each division represents 500 milliseconds. Pulse group PG is the difference between time X4 and X3, which in this graph is about 1.15 seconds.

Each pulse P with pulse group PG has a maximum of 19.3 Vdc. Furthermore, each pulse group PG alternates such that the first pulse group PG has a positive voltage, while the next pulse group PG has a negative voltage. This embodiment equates to electrodes 202 and 204 having alternating currents.

While these maximum output voltages, maximum output current, net charge, maximum current density, and maximum power density are typical for device 100, these statistics should not be construed as limiting to the scope of this application.

Returning to FIG. 5, lines 506 and 508 couple processor 412 to electromagnetic radiation component 208. Electromagnetic radiation component 208, in this embodiment, incorporates 12 evenly spaced light emitting diodes (LEDs) 510 around the common center shared by electrodes 202 and 204. LEDs 510 are semiconductor devices that emit light when electrical current is applied in the forward direction by line 506 and ending on line 508.

LEDs 510 can emit varying wavelengths of light dependent on the composition and condition of the semiconducting material used. LEDs 510 can emit infrared, visible, or ultraviolet light. While in this embodiment electromagnetic radiation component 208 incorporates LEDs 510, electromagnetic radiation component 208 can also include other forms of electromagnetic radiation.

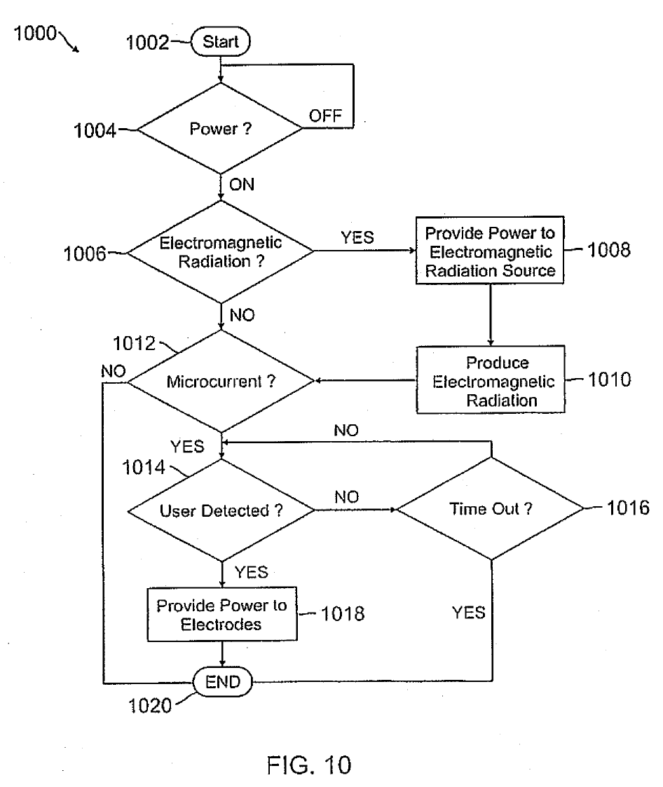

FIG. 10 represents an exemplary flow chart illustrating method 1000 used by device 100 to provide microcurrent and photorejuvenation therapies. Method 1000 begins at position 1002. At decision block 1004, device 100 determines whether power button 210 has been pressed and the power is on. When the power is off, control is returned to decision block 1004.

When button 210 has been pressed and the power is on, device 100 determines whether button 214 has been pressed at decision block 1006. When selected, power is provided to the electromagnetic radiation source at block 1008. In turn, the electromagnetic radiation source produces electromagnetic radiation at block 1010 and control is returned to decision block 1012.

Control is given to decision block 1012 when button 214 has not been selected or control is given by block 1010. At block 1012, device 100 determines whether button 212 has been selected. When not selected, method 1000 ends at position 1020.

If, however, the user has selected button 212, device 100 determines whether a user is detected at decision block 1014. Control is sent to decision block 1016 when no user has been detected. At decision block 1016, device 100 determines whether device 100 has timed out. If device 100 has timed out, method 1000 ends at position 1020. If not, control is given back to decision block 1014.

When a user has been detected by device 100, power is provided to electrodes 202 and 204 so that current may flow between them. Once completed, method 1000 ends at position 1020.

In accordance with another aspect of the present application, a method for treating skin 102 conditions using device 100 is presented. The method determines at least one type of treatment selected by the user.

When the selected treatment includes microcurrent therapy, the method applies current to the user through two or more electrodes 202 and 204. The two or more electrodes 202 and 204 are concentric circles separated by nonconducting material 206. The nonconducting material 206 prevents current from passing between the two or more electrodes 202 and 204 even when the power is on. Current passes between the two or more electrodes 202 and 204 when the two or more electrodes 202 and 204 contact the user. Through this medium or surface (i.e. the user), current can flow between electrodes 202 and 204 and around nonconducting material 206.

When photorejuvenation therapy is selected by the user, the method applies light to the user through a light source. Typically, the light source generates light having a wavelength of about 630 nanometers. The light source generally surrounds the two or more electrodes 202 and 204, the light source itself being a concentric circle.

In accordance with yet another aspect of the present application, a method for treating and preventing skin 102 conditions is presented. The method includes selecting at least one type of treatment through the user interface of device 100.

The method includes placing two or more electrodes 202 and 204 on a target area if the user selected microcurrent therapy. The two or more electrodes 202 and 204 are concentric circles with current passing between them through the user when the electrodes 202 and 204 contact the user.

If the user selected phototherapy, the method includes directing light to the target area. The user may also apply a topical treatment for enhancing the penetration of current and light into the user's skin 102.

In accordance with yet another aspect of the present application, a skin 102 care device 100 is presented. The device 100 includes a power source 406, at least one processor 412, and memory 416 operatively coupled to the processor 412.

The memory 416 stores program instructions that when executed by the processor 412, cause the processor 412 to perform at least one of the processes selected from the group below. The program instructions executed by the processor 412 can be selected through a user interface on device 100.

The group of processes includes a process for allocating power from the power source 406 to a circular cathode and anode having a common center point. Current passes from the cathode to the anode when the cathode and anode contact a user.

The group also includes a process for allocating power from the power source 406 to an electromagnetic radiation source 208. The electromagnetic radiation source 208 directs energy on the user.

Furthermore, the group includes a combination of the processes presented above. In additional embodiments, execution of the program instructions further cause the processor 412 to perform the process of detecting whether the two or more electrodes 202 and 204 are contacting the user.

Device 100 provided above can take the form of an entirely hardware embodiment or an embodiment containing both hardware and software elements. In one embodiment, device 100 uses software, which includes but is not limited to firmware, resident software, microcode, etc.

A data processing system suitable for storing and/or executing program code comprises at least one processor 412 coupled directly or indirectly to memory elements 416 through a system bus. The memory elements 416 can include local memory 416 employed during actual execution of the program code, bulk storage, and cache memories that provide temporary storage of at least some program code in order to reduce the number of times code is retrieved from bulk storage during execution. Input/output or I/O devices can be coupled to device 100 either directly or through intervening I/O controllers.

The foregoing description is provided to enable any person skilled in the relevant art to practice the various embodiments described herein. Various modifications to these embodiments will be readily apparent to those skilled in the relevant art, and generic principles defined herein may be applied to other embodiments. Thus, the claims are not intended to be limited to the embodiments shown and described herein, but are to be accorded the fill scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the art are expressly incorporated herein by reference and intended to be encompassed by the claims. Moreover, nothing disclosed herein is intended to be dedicated to the public regardless of whether such disclosure is explicitly recited in the claims.

What is claimed is:

1. A system for treating and preventing skin conditions on a user comprising;
a power source;
a power management component for allocating power from the power source;
a cathode and anode having a common center point, wherein the power management component supplies power to the cathode and anode when microcurrent therapy is desired;
an insulator between the cathode and anode inhibiting electric charge from flowing between the cathode and anode, current capable of passing between the cathode and anode when both the cathode and anode contact the user; and
a light source surrounding the cathode and anode, wherein the power management component supplies power to the light source when phototherapy is desired, the light source capable of directing light towards the user.

2. The system of claim 1, further comprising a handheld housing enclosure unit having a body and head.

3. The system of claim 2, wherein the body of the handheld housing includes the power source and power management component.

4. The system of claim 2, wherein the head of the handheld housing includes the cathode, anode, insulator, and light source.

5. The system of claim 2, wherein the head can pivot in multiple directions.

6. The system of claim 2, wherein the head of the handheld housing is interchangeable.

7. The system of claim 1, further comprising a user interface for allowing the user to select the desired therapy.

8. The system of claim 1, wherein the light source comprises a plurality of LEDs evenly spaced around the common center point.

9. An apparatus for preventing and treating skin conditions, the apparatus comprising;
two or more electrodes, wherein the two or more electrodes are concentric circles;
nonconducting material separating the two or more electrodes preventing current from passing between the two or more electrodes, wherein current passes between the two or more electrodes when the two or more electrodes contact the user an electromagnetic radiation source for providing light on the user.

10. The apparatus of claim 9, wherein the current between the two or more electrodes alternates.

11. The apparatus of claim 9, wherein a voltage between the two or more electrodes is regulated.

12. The apparatus of claim 9, wherein the current passes through the two or more electrodes at a frequency dependent on the user's type of skin tissue.

13. The apparatus of claim 9, wherein the surface area of the two or more electrodes are equivalent or nearly equivalent producing even current flow.

14. The apparatus of claim 9, wherein the nonconducting material is silicone.

15. The apparatus of claim 9, wherein the electromagnetic radiation source is light.

16. The apparatus of claim 15, wherein the light is made from a plurality of LEDs producing a wavelength of about 630 nanometers.

17. The apparatus of claim 9, wherein the electromagnetic radiation source is a concentric circle surrounding the two or more electrodes.

18. A skin care device comprising;
a power source;
at least one processor; and
a memory operatively coupled to the processor, the memory storing program instructions that when executed by the processor, cause the processor to perform at least one of the processes selected from the group consisting of:
allocate power from the power source to a circular cathode and anode having a common center point, whereby current passes from the cathode to the anode when the cathode and anode contact a user;
allocate power from the power source to an electromagnetic radiation source, the electromagnetic radiation directing energy on the user;
a combination thereof.

19. The skin care device of claim 18, wherein the program instructions executed by the processor are selected through a user interface.

20. The skin care device of claim 18, wherein the execution of the program instructions further cause the processor to perform the process of detecting whether the two or more electrodes are contacting the user.

21. A kit comprising:
a base unit; and
an apparatus for treating and preventing user skin conditions and capable of recharging when coupled to the base unit, the apparatus comprising:
two or more electrodes, wherein the two or more electrodes are concentric circles having a common center point;
a gasket separating the two or more electrodes preventing current from passing between the two or more electrodes, current passing between the two or more electrodes when the two or more electrodes contact the user;
an electromagnetic radiation source.

22. The kit of claim 21, further comprising a topical treatment for enhancing the penetration of current and electromagnetic radiation into the user's skin.

23. A method for treating skin conditions comprising:
determining at least one type of treatment selected by a user;
applying a current to the user through two or more electrodes if the selected treatment includes microcurrent therapy, wherein the two or more electrodes are concentric circles separated by a nonconducting material that prevents current from passing between the two or more electrodes, current passing between the two or more electrodes when the two or more electrodes contact the user; and
applying a light source on the user if the selected treatment includes photorejuvenation therapy.

24. The method of claim 23, wherein the current applied to the user adjusts at about 8.7 hertz.

25. The method of claim 23, wherein each pulse of the current lasts for about 57 milliseconds to about 58 milliseconds with each pulse group lasting about 1.15 seconds.

26. The method of claim 23, wherein applying a light source on the user comprises generating light having a wavelength of about 630 nanometers.

27. The method of claim 23, wherein the light source surrounds the two or more electrodes, the light source itself being a concentric circle.

28. A method for treating and preventing skin conditions on a user comprising:
selecting at least one type of therapy;
placing two or more electrodes on a target area if the user selected microcurrent therapy, wherein the two or more electrodes are concentric circles with current passing between the two or more electrodes through the user when the two or more electrodes contact the user; and
directing a light to the target area if the user selected phototherapy.

29. The method of claim 28, wherein selecting at least one type of therapy comprises choosing from a plurality of buttons located on a user interface, the buttons indicating whether the selected therapy has been chosen or not.

30. The method of claim 28, further comprising applying a topical treatment for enhancing the penetration of current and light into the user's skin.

31. A device for skin care using both microcurrents and light, the device comprising:
a power source;
a user interface for determining user selections;
two or more electrodes, wherein the two or more electrodes are concentric circles, power being supplied to the two or more electrodes from the power source when the user selects microcurrent therapy from the user interface;
insulating material separating the two or more electrodes preventing current from passing between the two or more electrodes, current passing between the two or more electrodes when the two or more electrodes contact the user; and
an electromagnetic radiation source, power being supplied to the electromagnetic radiation source from the power source when the user selects phototherapy from the user interface.

32. The device of claim 31, wherein the power source is rechargeable.

33. The device of claim 31, wherein the two or more electrodes comprise a cathode and anode, whereby current flows evenly from the cathode to the anode.

34. The device of claim 33, wherein the cathode is surrounded by the anode, charge flowing from an inward direction to an outward direction.

35. The device of claim 33, wherein the anode is surrounded by the cathode, charge flowing from an outward direction to an inward direction.

36. The device of claim 31, further comprising a power manager for storing power.

37. The device of claim 31, wherein the electromagnetic radiation source produces light.

38. A user skin care device comprising:
a microcurrent therapy component having two or more circular electrodes with a common center and capable of passing current between the two or more electrodes when the two or more electrodes contact the user; and
an electromagnetic radiation therapy component having a light source and capable of directing light towards the user.

39. An apparatus for treating and preventing skin conditions on a user, the apparatus comprising:
a power source;
a power management component for allocating power from the power source;
a user interface for determining user selections;
a cathode and anode, wherein the cathode and anode are concentric circles having a common center, the power management component supplying power to the cathode and anode when microcurrent therapy is selected from the user interface;
a nonconducting material between the cathode and anode preventing electric charge from flowing between the cathode and anode, current capable of passing between the cathode and anode through the user when both the cathode and anode contact the user;
the cathode and anode having nearly equivalent or equivalent surface areas producing even current flow;
a light source surrounding the cathode and anode, wherein the power management component supplies power to the light source when phototherapy is selected from the user interface, the light source capable of directing light towards the user; and
the light made from a plurality of LEDs producing a wavelength of about 630 nanometers.

40. The apparatus of claim 39, further comprising a detector for determining when the cathode and anode contact the user.
